(19) United States
(12) Patent Application Publication
(10) Pub. No.: US 2024/0351968 A1
(43) Pub. Date: Oct. 24, 2024

(54) CATALYTICAL PROCESS OF MAKING 4-HEXEN-3-ONE

(71) Applicant: INTERNATIONAL FLAVORS & FRAGRANCES INC., NEW YORK, NY (US)

(72) Inventor: Hongping XU, JianDe (CN)

(21) Appl. No.: 18/683,046

(22) PCT Filed: Mar. 18, 2022
(86) PCT No.: PCT/US2022/020995

§ 371 Date: Feb. 12, 2024

(30) Foreign Application Priority Data
Aug. 16, 2021 (CN) 202110940283.8

(51) Int. Cl. C07C 45/73; B01J 31/18

(52) U.S. Cl. CPC C07C 45/73; B01J 31/181; B01J 31/1815; B01J 2231/34; B01J 2531/004; B01J 2531/26

(57) ABSTRACT

This disclosure relates to a process which involves reacting 2-butanone with acetaldehyde in the presence of a zinc complex catalyst in a reaction zone to produce a product mixture comprising 4-hexen-3-one and 3-methyl-3-penten-2-one. This disclosure also relates to a process which involves (a) reacting 2-butanone with acetaldehyde in the presence of a zinc complex catalyst to produce a product mixture comprising 4-hexen-3-one, 3-methyl-3-penten-2-one and the zinc complex catalyst; (b) recovering the zinc complex catalyst from the product mixture; and (c) reusing the recovered zinc complex catalyst in the reacting step (a).

### BACKGROUND

### Field of the Disclosure

The present disclosure relates to the catalytical reactions of 2-butanone with acetaldehyde in the presence of zinc complex catalyst to make 4-hexen-3-one.

### Description of Related Art

4-hexen-3-one is an important intermediate for the production of methyl 2,5-dimethyl resorcylate which is an IFF (International Flavors & Fragrances, Inc.) Veramoss® fragrance product. Methyl 2,5-dimethyl resorcylate is a fragrance ingredient widely used in soaps, detergents and perfumes. Synthesis of 4-hexen-3-one has been reported. For example, CN103030541A disclosed a production method of 4-hexene-3-one by catalytical dehydration of 4-hydroxy-3-hexanone.

### BRIEF SUMMARY OF THE DISCLOSURE

The present disclosure provides a process for making 4-hexen-3-one (CH₃CH═CHC(O)CH₂CH₃). The process comprises: (a) reacting 2-butanone with acetaldehyde in the presence of a zinc complex catalyst in a reaction zone to produce a product mixture comprising 4-hexen-3-one and 3-methyl-3-penten-2-one (CH₃C(O)C(CH₃)═CHCH₃).

The present disclosure provides another process for making 4-hexen-3-one. The process comprises: (a) reacting 2-butanone with acetaldehyde in the presence of a zinc complex catalyst in a reaction zone to produce a product mixture comprising 4-hexen-3-one, 3-methyl-3-penten-2-one and the zinc complex catalyst; (b) recovering the zinc complex catalyst from the product mixture; and (c) reusing the recovered zinc complex catalyst in the reacting step (a).

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8”, “3 to 10”, “2 to 7”, “1.5 to 6”, “3.4 to 7.8”, “1 to 2 and 7-10”, “2 to 4 and 6 to 9”, “1 to 3.6 and 7.2 to 8.9”, “1-5 and 10”, “2 and 8 to 10”, “1.5-4 and 8”, and the like.

The present disclosure illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

Before addressing details of embodiments described below, some terms are defined or clarified.

The term “zinc complex”, as used herein, means a zinc coordination complex comprising a central zinc cation (Zn(II)) surrounded by one or more organic coordination ligands that bind to the central zinc cation. In the zinc complex, zinc is in an oxidation state of +2 (i.e., Zn(II)). The bonding with the zinc cation generally involves formal donation of one or more of the ligand's electron pairs.

The term “zinc acetate”, as used herein, means anhydrous and/or hydrated zinc acetate. In some embodiments, zinc acetate is zinc acetate dihydrate. In some embodiments, zinc acetate is anhydrous.

The present disclosure provides a process for making 4-hexen-3-one. The process comprises: (a) reacting 2-butanone with acetaldehyde in the presence of a zinc complex catalyst in a reaction zone to produce a product mixture comprising 4-hexen-3-one and 3-methyl-3-penten-2-one. In some embodiments, the process further comprises contacting a zinc compound with an organic ligand in the reaction zone to form the zinc complex catalyst in situ before the reacting step (a).

In the reaction zone, reactants 2-butanone and acetaldehyde are mixed with the zinc complex catalyst to form a reaction mixture. The zinc complex catalyst is not zinc oxide, zinc acetate, or zinc acetate dihydrate. In some embodiments, the zinc complex catalyst comprises Zn(II) cation and an organic ligand selected from the group consisting of pyridine, 2,2′-bipyridine, phenanthroline (1,10-phenanthroline), proline (pyrrolidine-2-carboxylic acid), salen (2,2′-ethylenebis(nitrilomethylidene)diphenol, or N,N′-bis(salicylidene)ethylenediamine), and combinations thereof. In some embodiments, the zinc complex catalyst comprises Zn(II) cation and an organic ligand selected from the group consisting of pyridine, 2,2′-bipyridine, phenanthroline, and combinations thereof. In some embodiments, the zinc complex catalyst comprises Zn(II) cation and pyridine. In some embodiments, the zinc complex catalyst comprises Zn(II) cation, pyridine and acetate. In some embodiments, the zinc complex catalyst comprises Zn(II) cation and 2,2′-bipyridine. In some embodiments, the zinc complex catalyst comprises Zn(II) cation, 2,2′-bipyridine and acetate. In some embodiments, the zinc complex catalyst comprises Zn(II) cation and phenanthroline. In some embodiments, the zinc complex catalyst comprises Zn(II) cation, phenanthroline and acetate.

In some embodiments, the zinc complex catalyst comprises no more than 30 wt % water, or no more than 25 wt % water, or no more than 20 wt % water, or no more than 15 wt % water, or no more than 10 wt % water, or no more than 5 wt % water, or no more than 2 wt % water, or no more than 1 wt % water, or no more than 0.5 wt % water, based on the total weight of the zinc complex catalyst and water content contained therein.

In some embodiments, the zinc complex catalyst is water-soluble. In some embodiments, the zinc complex catalyst has water solubility of at least 20 g/L (gram/liter), or at least 50 g/L, or at least 100 g/L, or at least 200 g/L, or at least 300 g/L, or at least 400 g/L, or at least 500 g/L, or at least 600 g/L, or at least 700 g/L, or at least 800 g/L, at 25° C., based on the volume of the aqueous solution of the zinc complex catalyst in water. In some embodiments, the zinc complex catalyst has water solubility of no more than 1200 g/L, or no more than 1500 g/L, or no more than 2000 g/L, or no more than 2500 g/L, or no more than 3000 g/L, or no more than 4000 g/L, at 25° C., based on the volume of the aqueous solution of the zinc complex catalyst in water.

In some embodiments, the zinc complex catalyst is made by contacting a zinc compound with an organic ligand. In some embodiments, the zinc complex catalyst can be made at room temperature. In some embodiments, a stoichiometrically excess amount of the zinc compound is used. In some embodiments, the mole ratio of the zinc compound to the organic ligand is at least 0.2, or at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4, or at least 1.5. In some embodiments, the mole ratio of the zinc compound to the organic ligand is no more than 5.0, or no more than 4.0, or no more than 3.0, or no more than 2.5, or no more than 2.0, or no more than 1.5, or no more than 1.2, or no more than 1.1, or no more than 1.0. In some embodiments, the mole ratio of the zinc compound to the organic ligand is from 0.5 to 1.5, or from 0.8 to 1.2.

In some embodiments, the zinc complex catalyst is made by contacting a zinc compound with an organic ligand in substantial absence of a solvent. In some embodiments, the amount of the solvent is no more than 10 wt %, or no more than 5 wt %, or no more than 2 wt %, or no more than 1 wt %, or no more than 0.5 wt %, or no more than 0.2 wt %, or no more than 0.1 wt %, based on the total weight of the zinc compound and the organic ligand. In some embodiments, the reaction mixture (formed by contacting the zinc compound with the organic ligand) comprises no more than 30 wt % water, or no more than 25 wt % water, or no more than 20 wt % water, or no more than 15 wt % water, or no more than 10 wt % water, or no more than 5 wt % water, or no more than 2 wt % water, or no more than 1 wt % water, or no more than 0.5 wt % water, based on the total weight of the zinc compound and the organic ligand.

In some embodiments, the zinc compound is selected from the group consisting of zinc oxide, zinc acetate, zinc chloride, zinc sulfate, and mixtures thereof. Zinc compound includes anhydrous and hydrated ones. In some embodiments, the zinc compound is zinc acetate (anhydrous or hydrated such as zinc acetate dihydrate). In some embodiments, the organic ligand is pyridine, 2,2′-bipyridine, phenanthroline, or mixtures thereof. In some embodiments, the organic ligand is 2,2′-bipyridine. In some embodiments, the organic ligand is phenanthroline.

In some embodiments, the zinc complex catalyst is made in situ. In such embodiments, a zinc compound and an organic ligand are fed into the reaction zone to form the zinc complex catalyst, preferably before the reaction of 2-butanone with acetaldehyde. In some embodiments, the zinc compound and the organic ligand are fed into the reaction zone before acetaldehyde is fed. When the zinc complex catalyst is made in situ, the process comprises (a1) contacting a zinc compound with an organic ligand in a reaction zone to form a zinc complex catalyst; and (a2) reacting 2-butanone with acetaldehyde in the reaction zone in the presence of the zinc complex catalyst to produce a product mixture comprising 4-hexen-3-one and 3-methyl-3-penten-2-one.

The starting materials 2-butanone and acetaldehyde can be fed into the reaction zone together or separately. The reaction between 2-butanone and acetaldehyde in this disclosure is based on a stoichiometry of 1 mole of 2-butanone per mole of acetaldehyde. In practice, an excess of 2-butanone may be used as desired. Typically, the mole ratio of 2-butanone to acetaldehyde fed into the reaction zone is from about 1:1 to about 10:1, or from about 1.2:1 to about 5:1, or from about 1.2:1 to about 3:1, or from about 1.5:1 to about 2:1. In some embodiments, the upper limit of the mole ratio is 10, 9, 8, 7, 6, 5, 4, 3, 2, 1.8, 1.6, or 1.4. In some embodiments, the lower limit of the mole ratio is 0.8, 0.9, 1.0, 1.1, 1.2, 1.3, 1.4, or 1.5.

In some embodiments, 2-butanone is first fed into the reaction zone, and then acetaldehyde is fed into the reaction zone at the reaction temperature continuously or in portions. In some embodiments, a portion of the 2-butanone is first fed into the reaction zone, and then a mixture of acetaldehyde and the rest of the 2-butanone is fed into the reaction zone at the reaction temperature continuously or in portions.

In some embodiments, the mole ratio of 2-butanone (fed into the reaction zone) to zinc complex catalyst (fed into the reaction zone or made in situ) is from about 10 to about 50, or from about 15 to about 45, or from about 20 to about 40. In some embodiments, the mole ratio of 2-butanone to zinc complex catalyst is at least 10, or at least 15, or at least 20, or at least 25, or at least 30. In some embodiments, the mole ratio of 2-butanone to zinc complex catalyst is no more than 50, or no more than 45, or no more than 40.

In some embodiments, the zinc complex catalyst is made in situ, and the mole ratio of 2-butanone (fed into the reaction zone) to zinc compound (e.g., zinc acetate, fed into the reaction zone) is from about 10 to about 50, or from about 15 to about 45, or from about 20 to about 40. In some embodiments, the mole ratio of 2-butanone to zinc compound is at least 10, or at least 15, or at least 20, or at least 25, or at least 30. In some embodiments, the mole ratio of 2-butanone to zinc compound is no more than 50, or no more than 45, or no more than 40.

In some embodiments, the reaction of 2-butanone with acetaldehyde is carried out in substantial absence of a solvent. In some embodiments, the amount of the solvent is no more than 10 wt %, or no more than 5 wt %, or no more than 2 wt %, or no more than 1 wt %, or no more than 0.5 wt %, or no more than 0.2 wt %, or no more than 0.1 wt %, based on the total weight of the reaction mixture. In some embodiments, essentially no additional water is added into the reaction zone during the reaction. By “additional water”, it is meant herein water in addition to or other than ones carried by reactants (2-butanone and acetaldehyde), zinc compound, organic ligand, and/or the zinc complex catalyst. For example, zinc acetate dihydrate carries hydrate water. In some embodiments, the reaction mixture comprises no more than 30 wt % water, or no more than 25 wt % water, or no more than 20 wt % water, or no more than 15 wt % water, or no more than 10 wt % water, or no more than 8 wt % water, or no more than 6 wt % water, or no more than 4 wt % water, or no more than 2 wt % water, based on the total weight of the reaction mixture.

Typically, the process of this disclosure is conducted at a temperature (reaction temperature, temperature in the reaction zone) of from about 100° C. to about 200° C., or from about 120° C. to about 180° C., or from about 150° C. to about 175° C., or from about 160° C. to about 165° C. In some embodiments, the reaction temperature is at least 100° C., or at least 110° C., or at least 120° C., or at least 130° C., or at least 140° C., or at least 145° C., or at least 150° C., or at least 155° C., or at least 160° C. In some embodiments, the reaction temperature is no more than 200° C., or no more than 190° C., or no more than 185° C., or no more than 180° C., or no more than 175° C., or no more than 170° C., or no more than 165° C.

The process of this disclosure can be conducted under a pressure (reaction pressure, pressure in the reaction zone) of from about 5 bar to about 15 bar, or from about 6 bar to about 12 bar, or from about 6 bar to about 10 bar. In some embodiments, the reaction pressure is at least 2 bar, or at least 3 bar, or at least 4 bar, or at least 5 bar, or at least 6 bar, or at least 7 bar. In some embodiments, the reaction pressure is no more than 25 bar, or no more than 20 bar, or no more than 15 bar, or no more than 12 bar, or no more than 10 bar, or no more than 8 bar. The process of this disclosure can be conducted in the presence of air.

Reaction time for the process of this disclosure can range from about 4 hours to about 20 hours, or from about 6 hours to about 16 hours, or from about 8 hours to about 12 hours. In some embodiments, the reaction time is at least 2 hours, or at least 3 hours, or at least 4 hours, or at least 5 hours, or at least 6 hours, or at least 7 hours, or at least 8 hours. In some embodiments, the reaction time is no more than 30 hours, or no more than 25 hours, or no more than 20 hours, or no more than 15 hours, or no more than 12 hours.

The reaction of 2-butanone with acetaldehyde in the process of this disclosure generates a product mixture comprising 4-hexen-3-one and 3-methyl-3-penten-2-one. The product mixture also comprises the zinc complex catalyst. The product mixture can be cooled (e.g., to room temperature) and form an organic phase and an aqueous phase. The organic phase comprises 4-hexen-3-one and 3-methyl-3-penten-2-one. In some embodiments, the organic phase also comprises unreacted reactants such as 2-butanone. The desired product 4-hexen-3-one can be separated and recovered by methods known in the art such as distillation. In some embodiments, the yield of 4-hexen-3-one is from about 16% to about 32%, or from about 18% to about 30%, or from about 20% to about 28%.

Typically, the aqueous phase comprises an aqueous solution with the zinc complex catalyst dissolved therein. In some embodiments, substantially no water is added to the product mixture or the reaction zone after the reaction to form the aqueous phase. In some embodiments, substantially no water is added to the aqueous phase.

In some embodiments, the zinc complex catalyst is recovered and reused. In such embodiments, the process of this disclosure comprises: (a) reacting 2-butanone with acetaldehyde in the presence of a zinc complex catalyst in a reaction zone to produce a product mixture comprising 4-hexen-3-one, 3-methyl-3-penten-2-one and the zinc complex catalyst; (b) recovering the zinc complex catalyst from the product mixture (to generate a recovered zinc complex catalyst); and (c) reusing the recovered zinc complex catalyst (from step (b)) in the reacting step (a), that is, reacting 2-butanone with acetaldehyde in the presence of the recovered zinc complex catalyst to produce a product mixture comprising 4-hexen-3-one, 3-methyl-3-penten-2-one and the recovered zinc complex catalyst. In some embodiments, the yield of 4-hexen-3-one in step (c) is substantially the same as the yield of 4-hexen-3-one in step (a). In some embodiments, the yield of 4-hexen-3-one for reaction of step (c) is within the range of ±10%, or ±15%, or ±20%, or ±25%, or ±30% from the yield of 4-hexen-3-one for reaction of step (a). In some embodiments, the recovered zinc complex catalyst can be reused without further purification (e.g., washing, dissolving in a solvent and then reprecipitating, etc.). In some embodiments, the zinc complex catalyst can be recovered and reused for twice or more times, that is, the steps (b) and (c) can be repeated for at least twice, or at least three times, or at least four times, or at least five times, or at least six times, or at least seven times, or at least eight times, or at least nine times, or at least ten times. The steps (b) and (c) can be repeated for many times as long as the yield of 4-hexen-3-one does not drop significantly. In some embodiments, the steps (b) and (c) are repeated for no more than forty times, or no more than thirty times, or no more than twenty-five times, or no more than twenty times, or no more than fifteen times, or no more than fourteen time, or no more than thirteen times, or no more than twelve times, or no more than eleven times, or no more than ten times, or no more than nine times, or no more than eight times.

One advantage of the present invention is that the yield of 4-hexen-3-one remains substantially constant for each reactions when the steps (b) and (c) are repeated for multiple times (e.g., at least ten times). In some embodiments, the yield of 4-hexen-3-one remains within the range of from about 18% to about 30%, or from about 20% to about 28%, for each reactions when the steps (b) and (c) are repeated. In some embodiments, the yield of 4-hexen-3-one for each reactions of step (c) is within the range of ±10%, or ±15%, or ±20%, or ±25%, or ±30% from the yield of 4-hexen-3-one for reaction of step (a). In some embodiments, the yield of 4-hexen-3-one for each reactions of step (c) is at least 14%, or at least 15%, or at least 16%, or at least 17%, or at least 18%, or at least 19%, or at least 20%, or at least 21%, or at least 22%, and no more than 35%, or no more than 30%, or no more than 28%, or no more than 26%.

In some embodiments, the zinc complex catalyst is recovered from the product mixture by removing water from the aqueous phase. Water can be removed from the aqueous phase by methods known in the art, such as evaporation. In some embodiments, at least 70%, or at least 75%, or at least 80%, or at least 85%, or at least 90%, or at least 92%, or at least 95% of the zinc complex catalyst, based on the amount of the zinc complex catalyst fed into the reaction zone or formed in situ in the reaction zone, can be recovered from the product mixture. In some embodiments, the recovered zinc complex catalyst comprises no more than 10 wt % water, or no more than 8 wt % water, or no more than 6 wt % water, or no more than 4 wt % water, or no more than 2 wt % water, or no more than 1 wt % water, based on the total weight of the recovered zinc complex catalyst and the water content contained therein.

3-Methyl-3-penten-2-one can be used as an intermediate for the production of (1S,4aS,8aS)-Decahydro-5,5,8a-trimethyl-2-methylene-1-naphthaleneacetaldehyde which is an IFF Iso E Super® fragrance product. In some aspects, the present disclosure also provides a process for the co-production of 4-hexen-3-one and 3-methyl-3-penten-2-one. The process comprises: (a) reacting 2-butanone with acetaldehyde in the presence of a zinc complex catalyst in a reaction zone to produce a product mixture comprising 4-hexen-3-one and 3-methyl-3-penten-2-one. 3-Methyl-3-penten-2-one can be separated and recovered from the product mixture by methods known in the art such as distillation. In some embodiments, the yield of 3-methyl-3-penten-2-one is from about 16% to about 32%, or from about 18% to about 30%, or from about 20% to about 28%.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

### Example 1

Into a 0.2 L autoclave fitted with a mechanical stirrer were added 17 g 2-butanone, 3 g 2,2′-bipyridine, and 4.5 g zinc acetate dihydrate. The reactants in the autoclave were stirred and heated to 160° C. Into a 0.05 L flask were added 17 g acetaldehyde and 26 g 2-butanone. The mixture of acetaldehyde and 2-butanone in the flask was added into the autoclave through a pump in a time period of 4 hours. The autoclave pressure was 8 bar. The autoclave temperature was then kept at 160° C. for 4 hours before cooled to room temperature. The reaction mixture was separated into two phases: an aqueous phase and an organic phase. The aqueous phase was separated from the organic phase, concentrated and used as the zinc complex catalyst for Example 2. The yield of 4-hexen-3-one was about 24% and the yield of 3-methyl-3-penten-2-one was about 24%.

### Example 2

Into a 0.2 L autoclave fitted with a mechanical stirrer were added 17 g 2-butanone and about 8 g zinc complex catalyst recovered from Example 1. The reactant and catalyst in the autoclave were stirred and heated to 160° C. Into a 0.05 L flask were added 17 g acetaldehyde and 26 g 2-butanone. The mixture of acetaldehyde and 2-butanone in the flask was added into the autoclave through a pump in a time period of 4 hours. The autoclave pressure was 8 bar. The autoclave temperature was then kept at 160° C. for 5 hours before cooled to room temperature. The reaction mixture was separated into two phases: an aqueous phase and an organic phase. The aqueous phase was separated from the organic phase, concentrated and used as the zinc complex catalyst for the next batch. The yield of 4-hexen-3-one was about 24% and the yield of 3-methyl-3-penten-2-one was about 24%.

### Example 3

Into a 0.2 L autoclave fitted with a mechanical stirrer were added 17 g 2-butanone, 3.6 g phenanthroline, and 4.5 g zinc acetate dihydrate. The reactants in the autoclave were stirred and heated to 160° C. Into a 0.05 L flask were added 17 g acetaldehyde and 26 g 2-butanone. The mixture of acetaldehyde and 2-butanone in the flask was added into the autoclave through a pump in a time period of 4 hours. The autoclave pressure was 8 bar. The autoclave temperature was then kept at 160° C. for 4 hours before cooled to room temperature. The reaction mixture was separated into two phases: an aqueous phase and an organic phase. The aqueous phase was separated from the organic phase, concentrated and used as the zinc complex catalyst for another reaction. The yield of 4-hexen-3-one was about 24% and the yield of 3-methyl-3-penten-2-one was about 24%.

### Example 4

Into a 0.2 L autoclave fitted with a mechanical stirrer were added 17 g 2-butanone, 2 g pyridine, and 4.5 g zinc acetate dihydrate. The reactants in the autoclave were stirred and heated to 160° C. Into a 0.05 L flask were added 17 g acetaldehyde and 26 g 2-butanone.

The mixture of acetaldehyde and 2-butanone in the flask was added into the autoclave through a pump in a time period of 4 hours. The autoclave pressure was 8 bar. The autoclave temperature was then kept at 160° C. for 4 hours before cooled to room temperature. The reaction mixture was separated into two phases: an aqueous phase and an organic phase. The aqueous phase was separated from the organic phase, concentrated and used as the zinc complex catalyst for Example 5. The yield of 4-hexen-3-one was about 16% and the yield of 3-methyl-3-penten-2-one was about 16%.

### Example 5

Into a 0.2 L autoclave fitted with a mechanical stirrer were added 17 g 2-butanone and 7 g zinc complex catalyst (containing about 10 wt % water content) recovered from Example 4. The reactant and catalyst in the autoclave were stirred and heated to 160° C. Into a 0.05 L flask were added 17 g acetaldehyde and 26 g 2-butanone. The mixture of acetaldehyde and 2-butanone in the flask was added into the autoclave through a pump in a time period of 4 hours. The autoclave pressure was 8 bar. The autoclave temperature was then kept at 160° C. for 5 hours before cooled to room temperature. The reaction mixture was separated into two phases: an aqueous phase and an organic phase. The aqueous phase was separated from the organic phase, concentrated and used as the zinc complex catalyst for the next batch. The yield of 4-hexen-3-one was about 8% and the yield of 3-methyl-3-penten-2-one was about 8%.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

1. A process comprising: (a) reacting 2-butanone with acetaldehyde in the presence of a zinc complex catalyst in a reaction zone to produce a product mixture comprising 4-hexen-3-one and 3-methyl-3-penten-2-one.

2. The process of claim 1, wherein the zinc complex catalyst comprises Zn(II) and an organic ligand selected from the group consisting of pyridine, 2,2′-bipyridine, phenanthroline, proline, salen, and combinations thereof.

3. The process of claim 2, wherein the zinc complex catalyst comprises Zn(II) and an organic ligand selected from the group consisting of pyridine, 2,2′-bipyridine, phenanthroline, and combinations thereof.

4. The process of claim 1 further comprising contacting a zinc compound with an organic ligand in the reaction zone to form the zinc complex catalyst in situ.

5. The process of claim 4, wherein the zinc compound is selected from the group consisting of zinc oxide, zinc acetate, zinc chloride, zinc sulfate, and mixtures thereof.

6. The process of claim 5, wherein the zinc compound is zinc acetate.

7. The process of claim 1, wherein reaction temperature is from about 150° C. to about 175° C.

8. The process of claim 1, wherein the mole ratio of 2-butanone to acetaldehyde fed into the reaction zone is from about 1.2:1 to about 2:1.

9. The process of claim 1, wherein the reacting step (a) is conducted in substantial absence of a solvent.

10. The process of claim 1, further comprising:
(b) recovering the zinc complex catalyst from the product mixture; and
(c) reusing the recovered zinc complex catalyst in the reacting step (a).

11. The process of claim 1, wherein the zinc complex catalyst comprises Zn(II) and an organic ligand selected from the group consisting of 2,2′-bipyridine, phenanthroline, and combinations thereof.

12. The process of claim 1, wherein the zinc complex catalyst comprises Zn(II) and 2,2′-bipyridine.

13. The process of claim 10, wherein the yield of 4-hexen-3-one in step (c) is substantially the same as the yield of 4-hexen-3-one in step (a).

14. The process of claim 1, wherein 4-hexen-3-one is recovered.

15. The process of claim 1, wherein 3-methyl-3-penten-2-one is recovered.
